(19) United States
(12) Patent Application Publication
(10) Pub. No.: US 2026/0137716 A1
(43) Pub. Date: May 21, 2026

(54) ENHANCED COMPOSITION FOR TREATMENT OF DENGUE FEVER

(71) Applicant: Frederick Sawaya, Lathrup Village, MI (US)

(72) Inventor: Frederick Sawaya, Lathrup Village, MI (US)

(21) Appl. No.: 19/261,061

(22) Filed: Jul. 7, 2025

Related U.S. Application Data
(60) Provisional application No. 63/721,664, filed on Nov. 18, 2024.

(51) Int. Cl. A61K 33/04; A61K 31/375; A61K 31/616; A61K 33/06; A61K 33/30

(52) U.S. Cl. CPC A61K 33/04; A61K 31/375; A61K 31/616; A61K 33/06; A61K 33/30

(57) ABSTRACT

A combination of sodium thiosulfate, zinc and magnesium and, optionally, Vitamin C is used to treat dengue fever. The components may be used or ingested or administered either simultaneously or sequentially in about 30 minutes separate administration. These Supplements are used to replace the zinc and magnesium which may be depleted from the body during treatment. Also, Vitamin C is used as an anti-viral composition. In cases where the patient is critically ill, a quantity of acetylsalicylic acid is administered.

### CROSS-REFERENCE TO RELATED APPLICATION

This Application is a Completion Application of Co-pending U.S. Provisional Patent Application Ser. No. 63/721,664, filed on Nov. 18, 2024 for Enhanced Composition For Treatment of Dengue Fever, the disclosure of which is hereby incorporated by reference.

### PRIOR ART

The present invention pertains to therapeutic compositions for the treatment, inhibition or attenuation of a virus and in particular, the dengue fever virus.

In U.S. Pat. No. 9,694,030, the disclosure of which is hereby incorporated by reference in its entirety, including the drawing, there is taught a composition principally based upon the utilization of sodium thiosulfate for effective administration in treating viral infections, including the Ebola virus, herpes virus, dengue fever, smallpox, measles, mumps rubella, yellow fever, and the like. This '030 patent focuses mainly on the treatment of the Ebola virus and with sodium thiosulfate. The predicate for the Ebola virus treatment in the '030 patent, is that the sodium thiosulfate biodegrades amino acid, polypeptide proteins, and glycol proteins in a virus and releases, inter alia, hydrogen sulfur dioxide as a breakdown product. It is further theorized that the splitting of the sulfur-to-sulfur (S—S) bond in the biodegrading of the glycol protein enables the thiosulfate to destroy or disable the mucin protein in the Ebola virus.

Although, as noted above, the '030 Patent intimates that various other viruses can be disabled through the use of sodium thiosulfate, it has been found that with respect to certain specific viruses, and particularly dengue fever, it is beneficial to enhance the efficacy of the thiosulfate.

It is with this increased efficacy to which the present invention is directed.

### SUMMARY OF THE INVENTION

Dengue fever can be treated by administering an effective amount of sodium thiosulfate in conjunction with replacement of certain minerals and vitamins as described below. The present invention further contemplates that in critical conditions in patients, acetylsalicylic acid in effective amounts is administered. The present treatment contemplates daily treatment for predetermined time periods in effective quantities and in direct relation to the age and size of the patient.

### DETAILED DESCRIPTION OF THE INVENTION

In accordance with the present invention, a therapeutic effective amount of sodium thiosulfate for treatment of dengue fever is supplemented with necessary vitamins and minerals as well as with an effective amount of acetylsalicylic acid where the patient is severely critical to disable the virus.

As with the reasoning set forth in the '030 patent, it is believed that the thiosulfate destroys the S—S bond of the dengue fever virus and in doing so, disrupts the viral RNA, but not the human RNA. In other words, the thiosulfate deployed as a medically safe compound, has the capability of splitting the S—S bond in the virus into two of the three sulfur atoms from the STS, and such reaction, splits the bonds, digests protein amino acids and glycoproteins.

However, in disrupting the viral RNA, it also depletes the body of zinc, Vitamin C and magnesium. Thus, these depleted essential minerals and vitamins must be replaced to preclude a potentially deleterious side effect from occurring when treating the dengue fever with the sodium thiosulfate. These supplements are necessary in order to avoid side effects from the thiosulfate treatment. Therefore, zinc, Vitamin C and magnesium are sequentially administered with the administration of the thiosulfate.

It is believed that the disruption of the body's zinc due to dengue fever is more pronounced than in the Ebola virus, which necessitates zinc replacement. The same is true regarding magnesium.

Vitamin C is an antiviral vitamin which promotes the destruction of the virus.

Acetylsalicylic acid is believed to accelerate the activity of the thiosulfate but concomitantly accelerates the depletion of the magnesium and zinc. Therefore, it is employed only when the patient is critically ill. In most instances, it should only be used in adults.

In use, the thiosulfate, which is commercially available as an anhydrous powder, is administered generally two to three times per day as an aqueous solution containing from about 100 mg per ounce of water to about 800 mg to about 1,600 mg per ounce of water, depending upon the age and physical attributes of the patient.

Magnesium, which is used as an aqueous solution of magnesium sulfate, is generally used in an amount ranging from about 250 mg about 500 mg per dose, on a daily basis in order to replenish the amount depleted by the thiosulfate.

Vitamin C on the other hand, is an antiviral compound which enhances the efficacy of the utilization of the thiosulfate. Vitamin C will readily dissolve in about one ounce of water and is administered at a rate of 100 mg per ounce of water daily with each administered dosage.

In use, the zinc powder is readily dissolved in about one ounce of water and is used in an amount ranging from about 50 mg to about 100 mg per one ounce of water.

Similarly, a 300 mg adult dosage of acetylsalicylic acid will dissolve in one to two ounces of water, as well as the 84 mg or child's aspirin dosage, and is so-administered on a daily basis per dose.

Ordinarily, an effective treatment hereof is to administer the salicylic acid, if necessary, zinc and magnesium in conjunction with the thiosulfate two to three times per day. In administering the components, generally, they may be ingested in any order, i.e., simultaneously or sequentially. They may be taken individually about twenty minutes apart and about two to three times per day. Also, two of the three components may be taken at one time and the third about twenty minutes later.

Furthermore, the administration of the other components begins about three hours after the thiosulfate is introduced into the user's system.

It is also possible to incorporate excipients such as microcrystalline cellulose and the like into the treatment.

It should be noted that the lower levels of the salicylic acid is applicable to children, including the use of 84 mg aspirin.

It is important to note that the amount of the various components that comprise “effective” amounts are, generally, dictated by the age, gender and weight of the individual. Typically, the requisite amounts can be determined by reference to the published Recommended Dietary Allowances (RDA).

With respect to the present invention, the following sets forth the recommended daily dosage of components based on weight:

TABLE 1

| Weight (lbs.) | Amount in 2-4 oz. Water |
|---|---|
| 100 | ¼ tsp. daily |
| 100-150 | ½ tsp. daily |
| 150-175 | 1 tsp. daily |
| 175 and greater | 1 tsp. daily |

The administration of the components in the manner and mode described above, is carried out on a daily basis, as needed, usually for up to about thirty (30) days depending on the severity of the fever. The stated dosages for adults and children can be doubled or tripled depending on the severity of the fever.

It is possible for people in those geographic areas that have been affected by dengue fever to remain on the medication for life. In such a situation, the maintenance dosages would be ½ or ¼ of the dosages taken during treatment, but on a daily basis.

It is also important to note that during treatment, niacin is not to be taken less than three hours before or three hours after taking the thiosulfate.

What is claimed is:

1. A method for treatment, inhibition or attenuation of dengue fever in a patient in need thereof, the method comprising:
(a) administering an effective amount of sodium thiosulfate; and
(b) an effective amount of zinc and magnesium.

2. The method of claim 1 which further comprises administering a therapeutically effective amount of Vitamin C.

3. The method of claim 2, wherein:
a) the zinc is administered as an aqueous form of either zinc sulfate or zinc chloride;
b) the magnesium is administered as either a tablet or an aqueous solution of magnesium sulfate; and
c ) the Vitamin C is administered as a tablet or as an aqueous solution thereof.

4. The method of claim 1 which further comprises:
Administering an effective amount of acetylsalicylic acid.

5. The method of claim 4, wherein:
a) the thiosulfate is administered as an anhydrous powder in an amount of about 100 mg per ounce of water;
b) the acetylsalicylic acid is used in an amount ranging from about 84 mg to about 300 mg by weight and is administered as either a tablet or dissolved in water;
c) the zinc is present as an aqueous solution, in an amount ranging from about 50 mg to about 100 mg per ounce of water;
d) the magnesium is present in an amount ranging from about 250 mg to about 500 mg per ounce of water; and
e) the Vitamin C is administered in an amount ranging from about 700 mg to about 1,000 mg either as a tablet or liquid per dose.

6. The method of claim 4, wherein the acetylsalicylic acid is used as either a tablet or as an aqueous solution thereof.

7. The method of claim 1, wherein the thiosulfate is administered as an aqueous solution thereof.

8. The method of claim 1 wherein the thiosulfate and salicylic acid are administered sequentially, about two to three times per day, the salicylic acid being administered about 20 to 30 minutes after administering each dose of the thiosulfate.
